(19) United States
(12) Patent Application Publication
(10) Pub. No.: US 2026/0103435 A1
(43) Pub. Date: Apr. 16, 2026

(54) Methods of Synthesizing Corticiolic Acid

(71) Applicant: Amrita Vishwa Vidyapeetham, Coimbatore (IN)

(72) Inventors: Nanjan Pandurangan, The Nilgiris (IN); Sunil Kumar Deevi, Guntur (IN); Prasanna Ramani, Coimbatore (IN)

(73) Assignee: Amrita Vishwa Vidyapeetham, Coimbatore (IN)

(21) Appl. No.: 19/233,872

(22) Filed: Jun. 10, 2025

(30) Foreign Application Priority Data
Oct. 15, 2024 (IN) 202441078115

(51) Int. Cl. C07C 51/09; C07C 51/16

(52) U.S. Cl. CPC C07C 51/09; C07C 51/16; C07C 2523/44; C07C 2527/20

(57) ABSTRACT

The present invention discloses a method of synthesizing corticiolic acid. More particularly, cost-effective, and efficient methods for synthesizing corticiolic acid using different synthetic methodologies. Each method involves unique reaction conditions and steps, resulting in varying yields. All the methods avoid the use of hazardous pyrophoricreagents and BBr3 for deprotection. The invention provides an eco-friendly process for the synthesis of corticiolic acid and its derivatives.

### CROSS REFERENCE TO RELATED APPLICATIONS

The present application claims priority to an Indian Patent Application #202441078115 filed on 15 Oct. 2024.

### FIELD OF THE INVENTION

The present invention relates to methods of synthesizing corticiolic acid. More particularly, the present invention discloses methods for the synthesis of corticiolic acid, also known as 2,4-dihydroxy-6-pentadecylbenzoic acid, which is rapid and scalable. Said corticiolic acid possesses anti-diabetic activities.

### BACKGROUND OF THE INVENTION

Corticiolic acid, also known as 2,4-dihydroxy-6-pentadecylbenzoic acid, is a natural product, which is typically found in certain types of fungalmutants. It is known to be a vital precursor in the synthesis of other significant fungal metabolites such as Aquastatin-A, Aquastatin-B, Aquastatin-C, and Exophilic acid. These metabolites have been found to possess significant properties, making corticiolic acid a crucial compound in medicinal chemistry.

However, the extraction and synthesis of corticiolic acid from natural sources have been associated with several challenges. Reference is made to a non-patented document titled as “A study of cashew nutshell liquid purification and the synthesis of nonionic surfactants from the component phenols” by Ian Edward Bruce. The paper discloses the synthesis of the biosynthetic intermediate 2,4-dihydroxy-6-pentadecyl benzoic acid, by means of a Horner-Emmons modification to the Wittig reaction.

The natural method of extraction is extremely tedious and offers a very low yield. Moreover, the synthetic protocols reported for corticiolic acid synthesis involve complex procedures for the introduction of the pentadecyl side linker, which is an essential component in the structure of corticiolic acid. These methods often require the use of pyrophoric reagents, such as n-BuiLi, to generate LDA, which results in low yield and scalability limitations.

In addition, other synthesis methods require the use of Grignard reagents for the introduction of the side alkyl chainin to the core structure of the compound. Some protocols also involve the execution of a coupling reaction, specifically a Sonogashira coupling, followed by the reduction of unsaturated double bonds using Palladium on activated carbon through hydrogenation. These procedures involve the use of expensive and pyrophoric reagents, which further adds to the complexity and cost of the synthesis process.

Therefore, there is a need for a more efficient and less complex method for synthesizing corticiolic acid. The ideal method would avoid the use of pyrophoric reagents and complicated coupling reactions, would be scalable to allow for larger production volumes, and would result in a higher yield of corticiolic acid. Such a method would significantly improve the accessibility of corticiolic acid and its derived metabolites for various applications in the field of medicinal chemistry.

### OBJECTS OF THE INVENTION

In order to overcome the shortcomings in the existing state of the art, the present invention provides novel methods for synthesizing corticiolic acid, using different synthetic methodologies.

Yet another objective of the invention is to provide different methods for synthesizing corticiolic acid with varying yields.

Yet another objective of the invention is to provide novel methods for synthesizing corticiolic acid, a biologically active compound using different synthetic methods.

Yet another objective of the invention is to provide methods that are efficient, and scalable to multi-gram levels.

Another objective of the present invention is to provide a method for synthesizing corticiolic acid which devoid of the use of hazardous pyrophoric reagents for deprotection, making the synthetic process safer and more environmentally friendly.

Yet another objective of the present invention is to provide a method of synthesizing corticiolic acid which is optimized by reducing the steps of synthesis and requires minimum purification efforts, hence reducing the cost of overall synthesis.

### SUMMARY OF THE INVENTION

The present invention discloses novel methods for synthesizing corticiolic acid also known as 2,4-dihydroxy-6-pentadecylbenzoic acid, a pharmaceutical intermediate having anti-diabetic activity. According to one embodiment of the present invention, the method of synthesizing corticiolic acid involves several reaction steps with various reagents and conditions, yielding intermediates that are further reacted under various process parameters to yield corticiolic acid. The starting compound for this method is a bromo derivative, and the overall yield of corticiolic acid is in the range of 5%-10%. This method is devoid of the use of pyrophoric reagents and scalability difficulties.

According to one embodiment of the present invention, a different method of synthesizing corticiolic acid is presented. This method involves reacting pentadec-1-ene with various reagents under specific conditions to yield intermediates, which are then reacted to yield corticiolic acid. The overall yield of corticiolic acid in this method is 25%-41%.

Another embodiment of the present invention discloses a novel method of synthesizing corticiolic acid. This method involves reacting pentadec-1-ene with various reagents under specific conditions to yield a hydroxylated derivative, which is then reacted to yield corticiolic acid. The overall yield of corticiolic acid in this method is 40%-50% and the method avoids the use of BBr3 for deprotection, making it an eco-friendly process.

The present invention also provides a composition comprising corticiolic acid synthesized by any one of the methods described. The corticiolic acid in this composition can be used in biological studies and has potential antidiabetic activity. The corticiolic acid is one of the active scaffolds in the structure Aquastatin A, Band C. The corticiolic acid is obtained through a novel coupling step, adding value to the invention.

The foregoing paragraphs have been provided by way of general introduction and are not intended to limit the scope of the following invention. The described embodiments, together with further advantages, will be best understood by reference to the following detailed description taken in conjunction with the accompanying drawings.

### BRIEF DESCRIPTION OF DRAWINGS

FIG. 1 depicts a scheme 1 as synthetic pathway for the preparation of corticiolic acid from precursor compounds through various chemical reactions.

FIG. 2 depicts a scheme 2 as a schematic representation of the four-step synthetic pathway for corticiolic acid as described in Synthetic Methodology IL

FIG. 3 depicts a scheme 3 as synthetic pathway of corticiolic acid hydroxylated derivatives via cross-coupling, formylation, and oxidation reactions.

FIG. 4 depicts a scheme 4 as a general scheme for synthesis of corticiolic acid.

### DETAILED DESCRIPTION OF THE INVENTION

While the invention has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. Additionally, a reference to the singular includes a reference to the plural unless otherwise stated or inconsistent with the disclosure herein.

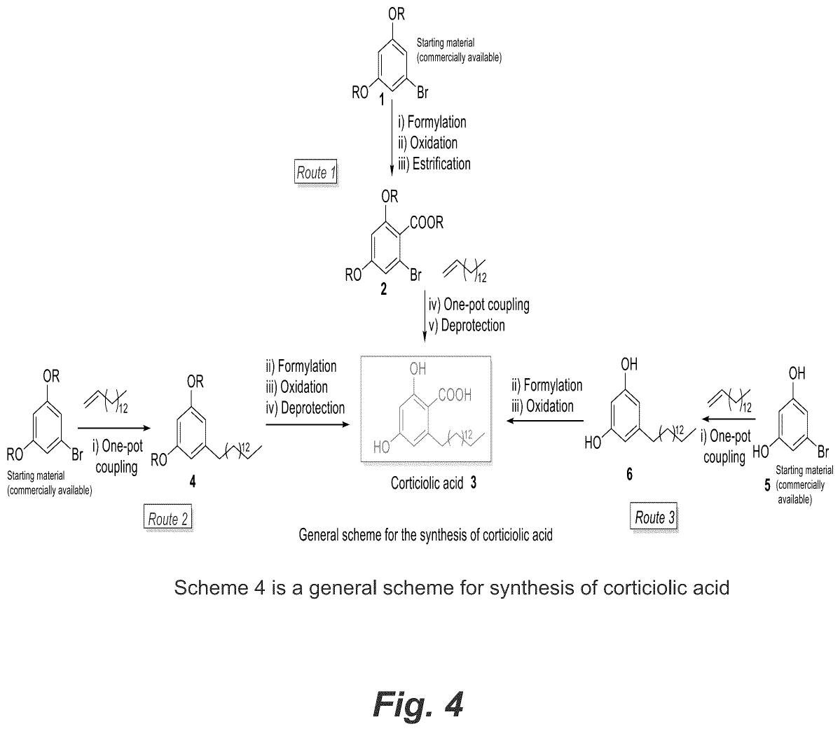

This invention presents three innovative methods for synthesizing corticiolic acid, a compound with potential applications in treating metabolic and viral diseases. The synthesis route of corticiolic acid was validated in three different route schemes where the number of steps was reduced from seven to three, FIG. 4 describes a general scheme of synthesis of corticiolic acid. The all route are novel and a shortened path for synthesizing corticiolic acid, which described as below:

### Route 1:

The present invention discloses a novel three-step process involving formylation, oxidation, and esterification of bromo dimethoxy. These processes are innovative in the synthesis of corticiolic acid. Similarly, the 9-BBN complex creation of pentadec-1-ene and bromo dimethoxy ester via an sp3-sp2 coupling provides a novel approach to corticiolic acid synthesis. This method avoided the use of pyrophoric reagents such as LDA and n-BuLi to introduce the alkyl chain. The reaction conditions are gentler and use easily available starting materials.

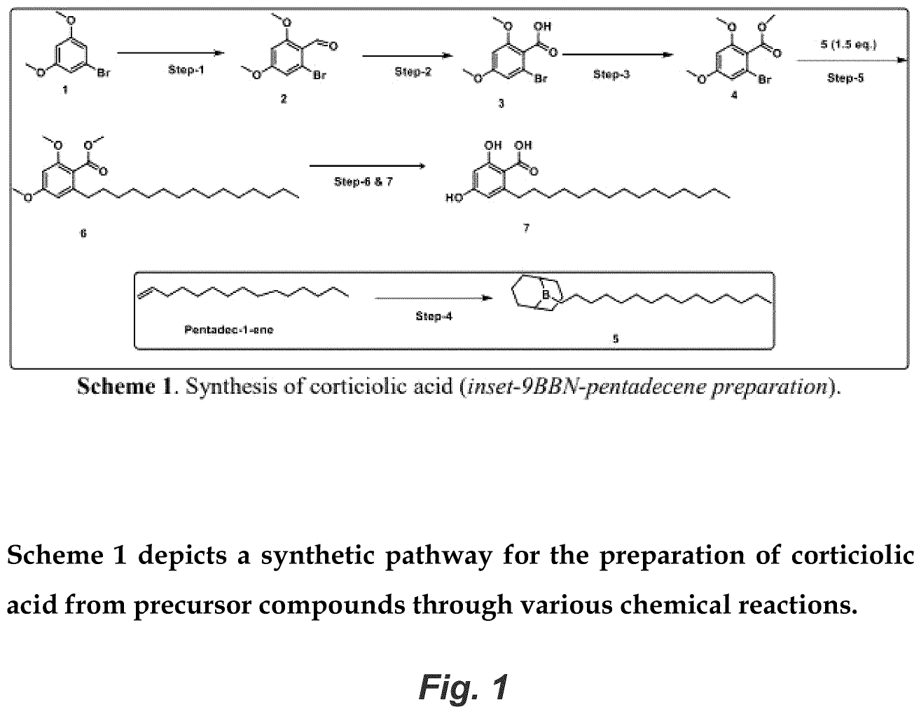

Methodology: Scheme 1 in FIG. 1 provides a detailed synthetic pathway for the preparation of corticiolic acid from precursor compounds through various chemical reactions. Said scheme 1 includes the synthesis of corticiolic acid as achieved by the first method. Reacting compound of formula 1 with POC13, DMF to yield compound of formula 2, reacting compound of formula 2 in presence of NaCl02, NaH2PO4, DMSO/H20 at 0° C. to room temperature to obtained compound of formula 3, reacting compound of formula 3 in presence of K2CO3, CH3I, DMF, at 50° C. to obtain compound of formula 4, then reacting compound of formula 4 with compound of formula 5, i.e., 9-BBN in presence of THF at 50° C. to obtain compound of formula 6, reacting compound of formula 6 with BBr3 to obtain corticiolic acid, 1.

The reaction conditions reported for the route 1 is milder with readily available starting materials. The synthetic route facilitates small scale to large scale starting from reported method is scalable and achieved in 5 g till 50 g of starting material.

### Route 2:

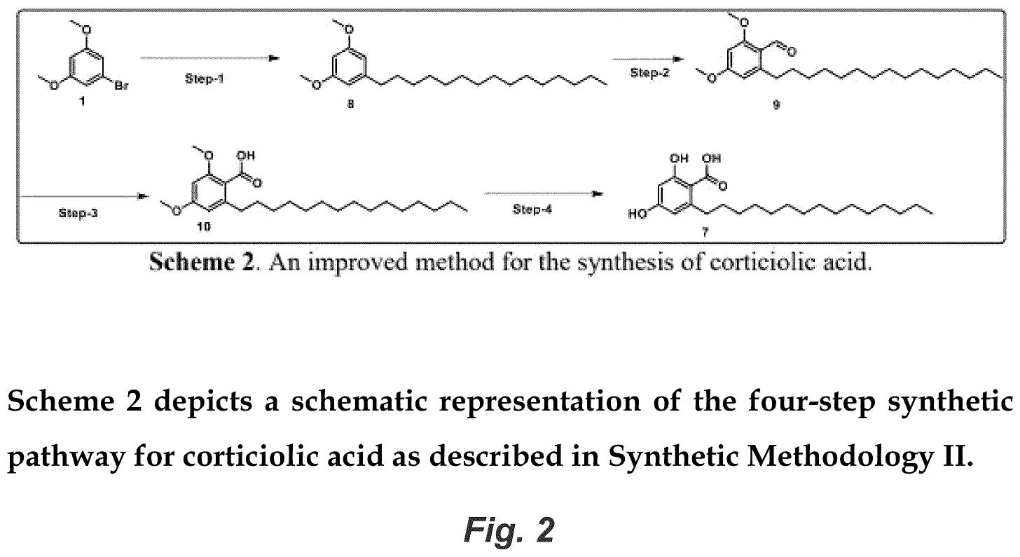

Another novel four-step process is depicted in FIG. 2 as scheme 2 involving formylation/oxidation/esterification/deprotection. Where sp3-sp2 coupling was carried out in the first step forming a key reaction. This four-step synthetic technique outperforms all other routes in terms of total yield up from 5% to 31% for corticiolic acid production. Scheme 2 provides a detailed synthetic pathway for the preparation of corticiolic acid from precursor compounds through various chemical reactions. Said scheme 2 includes the synthesis of corticiolic acid as achieved by the second method. Reacting compound 1 in the presence of pentadec-1-ene and 9-BBN, THE at 50° C., with K2CO3, Pd(dppf)Cl2DCM, DMF/Water, at 100° C. to yield compound 2, reacting compound 2 with POC13, DMF to yield compound 3. Compound 3 reacted with NaCl02, NaH2PO4, DMSO/H2O, at 0° C. to room temperature to obtain compound 4, reacting compound 4 with BBr3, DCM, at −78° C. to room temperature. The reaction was performed on a10 gm and which is scalable.

### Route 3

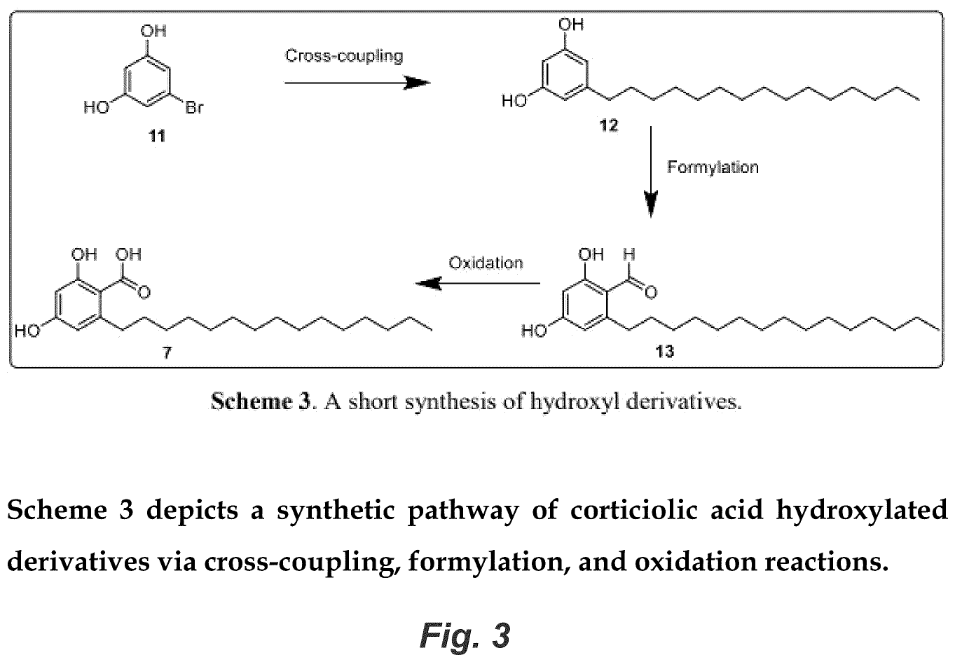

FIG. 3 depicts a method as scheme 3 that teaches the shortest route for the synthesis of corticiolic acid. In this method no protection or deprotection methods are used. As a result this method provides a quick synthesis of the natural substance corticiolic acid. Various techniques 1, 2, and 3 for producing corticiolic acid are scalable and detailed in multi-scale gram units (5-10 gm). Since there are no protection-deprotection processes required, thus this method avoids the use of harmful BBr3.

Methodology: Scheme 3 provides a detailed synthetic pathway for the preparation of corticiolic acid from precursor compounds through various chemical reactions. Said Scheme 3 includes the synthesis of corticiolic acid as achieved by the third method. Reacting compound 1 in the presence of pentadec-1-ene, 9-BBN, and THF at 50° C., for the Suzuki coupling with K2CO3, Pd(dppf)Cl2DCM, DMF/Water at 100 DC for synthesizing compound 2, reacting compound 2 with POC13, DMF to yield compound 3, reacting compound 3 in presence of NaClO2, NaH2PO4, DMSO/H2O, at O DC to room temperature to obtain compound 4.

This said method used to introduce side chains without reduction, ranging from the C9 to the C15 carbon chain. This innovative coupling step allowed us to obtain the saturated alkyl chain in a single step. Saturated alkyl side chain is produced in a single step by our method. Thus, common coupling phase was adjusted from low yield to high yield by changing the reactant/reagent/reaction parameter equivalents.

The methods disclosed in present invention minimize the synthetic processes, purification efforts, and overall cost of corticiolic acid synthesis over the course of the route1-route2-route-3 synthetic set. Overall, the present invention provides significant advancements in the synthesis of corticiolic acid and related compounds, offering promising potential for pharmaceutical and medicinal applications.

The novel coupling step enabled getting the saturated alkyl chain in one shot and avoiding further reduction of side chain double bond using various reducing agents which is a major disadvantage of the previously reported methods. This method yields saturated alkyl side chain in a one-step manner. The common coupling step was optimized from less yield to better yield by varying the equivalents of reactants/reagents/reaction parameters. The below Table 1 depicts the efforts of coupling condition optimization.

TABLE 1
Optimization of 9-BBN complex ofPentadec-1-ene and
Aryl halide SMC reaction for corticiolic acid.

| S. | | Isolated |
|---|---|---|
| No. | Reaction conditions | yield(%) |
| 1 | i) Pentadec-1-ene (1.0 eq.), 9-BBN (2.0 eq.), | 5% |
| | THF (5 v), RT, 16 h | |
| | ii) Cs2CO3(2.0 eq.), Pd(dppf)Cl2DCM (0.03 | |
| | eq.), DMF (10 v), sooc, 16 h | |
| 2 | i) Pentadec-1-ene(1.0 eq.), 9-BBN (2.0 eq.), | 13% |
| | THF (5 v), RT, 16 h | |
| | ii) K2CO3(2.0 eq.), Pd(dppt)Cl2DCM (0.03 eq.), | |
| | DMF/Water (9:1 v/v, 10 v), 80° C., 16 h | |
| 3 | i) Pentadec-1-ene (1.2 eq.), 9-BBN (3.0 eq.), | 21% |
| | THF (5 v), RT, 16 h | |
| | ii) K2CO3(2.0 eq.), Pd(dppt)Cl2DCM (0.03 eq.), | |
| | DMF/Water (9:1 v/v, 10 v), 80° c., 16 h | |
| 4 | i) Pentadec-1-ene (1.2 eq.), 9-BBN (3 eq.), | 45% |
| | THF (5 v), 50° C., 16 h | |
| | ii) K2CO3(2.5 eq.), Pd(dppt)Cl2DCM (0.05 eq.), | |
| | DMF/Water (9:1 v/v, 10 v), 80° C., 16 h | |
| 5 | i) Pentadec-1-ene (1.5 eq.), 9-BBN (5 eq.), | 78% |
| | THF (5 v), 50° C., 16 h | |
| | ii) K2CO3(2.5 eq.), Pd(dppt)Cl2DCM (0.05 eq.), | |
| | DMF/Water (9:1 v/v, 10 v), 100° c., 16 h | |
| END | iii) | |

We claim:

1. A method for synthesizing corticiolic acid, wherein said method of synthesis comprises the steps of
a) reacting a compound of formula 1 with phosphorus oxychloride (POCh) and dimethylformamide (DMF) to obtain a compound of formula 2;
b) reacting the compound of formula 2 with sodium chlorite (NaClO2), sodium dihydrogen phosphate (NalhPO4), and dimethyl sulfoxide (DMSO) in water at a temperature of O° C. to room temperature to obtain a compound of formula 3;
c) reacting the compound of formula 3 with potassium carbonate (KiCO3), methyl iodide (Cffil), and DMF at a temperature of 50° C. to obtain a compound of formula 4;
d) reacting the compound of formula 4 with compound of formula 5 in tetrahydrofuran (THF) at a temperature of 50° C. to obtain a compound of formula 6; and
e) reacting the compound of formula 6 with boron tribromide (BBn) to obtain corticiolic acid (compound of formula 7).

2. The claim as claimed in claim 1, wherein said compound of formula 5 is 9-BBN.

3. A method for synthesizing corticiolic acid, comprising the steps of:
a) reacting a compound of formula 1 with pentadec-1-ene and 9-borabicyclononane (9-BBN) in tetrahydrofuran (THF) at a temperature of 50° C., followed by reacting the resulting intermediate with potassium carbonate (KiCO3), palladium(II) dichloride (Pd(dppf)Ch) in dichloromethane (DCM), and dimethylformamide (DMF) in water at a temperature of 100° C. to obtain a compound of formula 2;
b) reacting the compound of formula 2 with phosphorus oxychloride(POCb) and dimethylformamide (DMF) to obtain a compound of formula 3;
c) reacting the compound of formula 3 with sodium chlorite (NaClOz), sodium dihydrogen phosphate (NaHzPO4), and dimethyl sulfoxide (DMSO) in water at a temperature of O° C. to room temperature to obtain a compound of formula 4; and
d) reacting the compound of formula (IV) with boron tribromide (BBr3) in dichloromethane(DCM) at a temperature of −78° C. to room temperature to obtain corticiolic acid (compound of formula 7.

4. The method as claimed in claim 1 wherein said step (a) is a sp3-sp2 coupling reaction.

5. A method for synthesis of corticiolic acid, wherein said method comprising the steps of:
a) reacting a compound of formula 1 with pentadec-1-ene, 9-borabicyclononane(9-BBN), and tetrahydrofuran(THF) at a temperature of 50° C., followed by reacting the resulting intermediate with potassium carbonate (K2CO3), palladium(II)dichloride (Pd(dppf)Clz) in dichloromethane (DCM), and dimethylformamide(DMF) in water at a temperature of 100° C. to obtain a compound of formula 2;
b) reacting the compound of formula 2 with phosphorus oxychloride(POCb) and dimethylformamide (DMF) to obtain a compound of formula 3;
c) reacting the compound of formula 3 with sodium chlorite (NaClOz), sodium dihydrogen phosphate (NaHzPO4), and dimethyl sulfoxide (DMSO) in water at a temperature of O° C. to room temperature to obtain a compound of formula 4; and
d)reacting the compound of formula 4with a reducing agent to obtain corticiolic acid (compound of formula 7).

6. The method of claim 3, wherein the reaction in step (a) is a Suzuki coupling reaction.

7. The method as claimed in claim 5, wherein the reducing agent is a hydrogenation catalyst.

8. The method as claimed in claim 5, wherein the hydrogenation catalyst is palladium on carbon (Pd/C).

9. The method of claimed in claim 3, wherein the compound of formula 1 is a precursor compound having a steroid structure.

10. The method as claimed in claim 1, wherein said method is scalable to multi-gram quantities.

11. The method as claimed in claim 1, wherein said corticiolic acid as synthesized possess potential antidiabetic activity.
